(19) United States
(12) Patent Application Publication
(10) Pub. No.: US 2023/0320667 A1
(43) Pub. Date: Oct. 12, 2023

(54) CONTACTLESS PHYSIOLOGICAL MEASUREMENT DEVICE AND METHOD

(71) Applicant: FACEHEART INC CORPORATION, Hsinchu City (TW)

(72) Inventors: Chun-Hsien Lin, Taoyuan City (TW); Yi-Chiao Wu, New Taipei City (TW); Meng-Liang Chung, Changhua City (TW); Bing-Fei Wu, Hsinchu City (TW)

(73) Assignee: FACEHEART INC CORPORATION, Hsinchu City (TW)

(21) Appl. No.: 17/715,289

(22) Filed: Apr. 7, 2022

(51) Int. Cl. A61B 5/00; A61B 5/024; A61B 5/021; A61B 5/0205; A61B 5/08; A61B 5/1455; G16H 50/30

(52) U.S. Cl. CPC A61B 5/6898; A61B 5/02427; A61B 5/021; A61B 5/7267; A61B 5/02405; A61B 5/0205; A61B 5/0816; A61B 5/14552; A61B 5/4872; G16H 50/30

(57) ABSTRACT

A contactless physiological measurement device is disclosed. The contactless physiological measurement device is an electronic device comprising a processor and a memory storing an application program. When the application program is executed, the processor controls a front camera and a rear camera of the electronic device to photograph a user, so as to obtain a face image and a hand image. Subsequently, after extracting a first rPPG signal and a second rPPG signal from the face image and the hand image respectively, physiological parameters are calculated by apply a signal process to the first/second rPPG signal. Moreover, a signal parameter difference is also acquired after applying a signal difference calculation to the two rPPG signals. Consequently, an estimation value of blood pressure is outputted by inputting user anthropometric parameter, said signal parameter difference and at least one physiological parameter into a pre-trained blood pressure estimating model.

### BACKGROUND OF THE INVENTION

### 1. Field of the Invention

The present invention relates to the technology field of physiological measurement apparatuses, and more particularly to a contactless physiological measurement device.

### 2. Description of the Prior Art

Human face is an important information source for a human being, e.g., a man commonly looks washed out after his illness. Therefore, the monitoring of physiological information is very important for assessing health and access to physiological data is not only necessary in clinical setting but it is becoming increasingly so also in other environments and applications related, for example, to telemedicine, personal fitness, e-commerce, trading and mental stress caused by the interaction with technology. Accordingly, an optical measuring technique called photoplethysmography (PPG) is developed and therefore used to measure the human pulse rate. Because the variations in blood volume are found to affect light transmission or reflectance accordingly, the principle of PPG measuring technique is to place an optical sensor directly on the skin of a subject, so as to detect the optical changes in blood volume. However, wearing the optical sensor leads specific subjects (especially infants and children) to feel uncomfortable and nervous, causing them have a negative feeling during the PPG-based physiological measurement.

To overcome the issue mentioned above, a remote photoplethysmography (rPPG) measuring technique is hence developed. Nowadays, rPPG is also known as imaging PPG (iPPG) or non-contact PPG (ncPPG), which allows contactless monitoring of human cardiac activity through a video camera, thereby further estimating various human physiological parameters, such as heart rate (HR), pulse rate, heart rate variability (HRV), blood pressure, respiratory rate, and blood oxygen saturation (SpO2). The information acquired through the rPPG measuring technique essentially refers to the cardiovascular functioning: the periodic blood flow and therefore the variations of blood volume in tissues that follow each cardiac cycle affects the optical properties of the tissues allowing those who are using this technology to measure at least one kind of physiological parameter remotely.

It is known that a rPPG measuring technique named “Facereader” is proposed by a software company named Noldus Information Technology in Netherlands. The Facereader rPPG system is able to monitor human physiological parameters through a patented rPPG measuring technique (EP2960862A). However, research reports have indicated that, the Facereader rPPG system tends to overestimate lower heart rate (HR) and underestimate higher HR compared to the traditional ECG system. These results suggest that the measurement accuracy of the conventional rPPG measuring technique still has room for improvement.

In view of this fact, inventors of the present application have made great efforts to make inventive research and eventually provided a contactless physiological measurement device.

### SUMMARY OF THE INVENTION

The primary objective of the present invention is to disclose a contactless physiological measurement device, which can be an electronic device comprising a processor, a memory, a front camera, and a rear camera, of which the memory stores an application program. When the application program is executed, the processor controls a front camera and a rear camera of the electronic device to photograph a user, so as to obtain a face image and a hand image. Subsequently, after extracting a first rPPG signal and a second rPPG signal from the face image and the hand image respectively, physiological parameters are calculated by apply a signal process to the first/second rPPG signal. Moreover, a signal parameter difference is also acquired after applying a signal difference calculation to the two rPPG signals. Consequently, an estimation value of blood pressure is outputted by inputting user anthropometric parameter, said signal parameter difference and at least one physiological parameter into a pre-trained blood pressure estimating model.

For achieving the primary objective mentioned above, the present invention provides an embodiment of the contactless physiological measurement device, which is provided in a form of an electronic device having a front camera and a rear camera, and comprising:
a processor, being coupled to the memory, and being also coupled to the front camera and the rear camera; wherein the application program includes instructions, such that in case the application program is executed, the processor being configured for:
acquiring, by controlling the front camera and the rear camera to photograph a user, at least one first image frame containing a face image and at least one second image frame containing a hand image;
extracting, from the face image of said first image frame and the hand image of said second image frame, respectively, a first rPPG signal and a second rPPG signal; acquiring, by applying a signal difference calculation to the first rPPG signal and the second rPPG signal, at least one signal parameter difference;
calculating, by applying a signal process to the first rPPG signal and the second rPPG signal, a plurality of physiological parameters; and
outputting, by inputting at least one user anthropometric parameter, at least one said physiological parameter and said signal parameter difference into a pre-trained blood pressure estimating model, an estimation value of blood pressure.

Moreover, the present invention also discloses a physiological measurement method, which is compiled to be an application program so as to be stored in a memory of an electronic device, and being conducted by a processor of the electronic device; the contactless physiological measurement method comprising the steps of:
(1) acquiring, by controlling a front camera and a rear camera of the electronic device to photograph a user, at least one first image frame containing a face image and at least one second image frame containing a hand image;
(2) extracting, from the face image of said first image frame and the hand image of said second image frame, respectively, a first rPPG signal and a second rPPG signal;
(3) calculating, by applying a signal difference calculation to the first rPPG signal and the second rPPG signal, at least one signal parameter difference;
(4) calculating, by applying a signal process to the first rPPG signal and the second rPPG signal, a plurality of physiological parameters; and
(5) outputting, by inputting at least one user anthropometric parameter, at least one said physiological parameter and said signal parameter difference into a pre-trained blood pressure estimating model, an estimation value of blood pressure.

In one embodiment, the electronic device is selected from a group consisting of smart phone, tablet computer, laptop computer, and all-in-one computer.

In one embodiment, the plurality of physiological parameters comprises heart rate (HR), heart rate variability (HRV), respiratory rate, and blood oxygen saturation (SpO2).

In one embodiment, the memory comprises a database storing at least one user anthropometric data and the plurality of physiological parameters.

In one embodiment, the application program consists of a plurality of subprograms, and the plurality of subprograms comprising:
a first subprogram, being compiled to be integrated in the application program by one type of programming language, and including instructions for configuring the processor to detect the face image from the first image frame;
a second subprogram, being compiled to be integrated in the application program by one type of programming language, and including instructions for configuring the processor to extract the first rPPG signal and the second rPPG signal from the face image of said first image frame and the hand image of said second image frame, respectively;
a third subprogram, being compiled to be integrated in the application program by one type of programming language, and including instructions for configuring the processor to apply said signal difference calculation to the first rPPG signal and the second rPPG signal, thereby calculating the at least one signal parameter difference; and
a fourth subprogram, being compiled to be integrated in the application program by one type of programming language, and including instructions for configuring the processor to input said user anthropometric parameter, said signal parameter difference and at least one said physiological parameter into the pre-trained blood pressure estimating model, such that the pre-trained blood pressure estimating model outputs said estimation value of blood pressure.

In one practicable embodiment, a time-domain signal is generated after applying said signal process to the first rPPG signal or the second rPPG signal, such that at least one time-domain parameter is allowed extracted from the time-domain signal; said time-domain parameter being selected from a group consisting of standard deviation of all normal to normal intervals (SDNN), root mean square successive differences (RMSSD), number of pairs of adjacent normal to normal intervals differing by more than 50 ms (NN50), proportion of NN50 divided by a total number of all normal to normal intervals (pNN50).

In another one practicable embodiment, a frequency-domain signal is generated after applying said signal process to the first rPPG signal or the second rPPG signal, such that at least one frequency-domain parameter is allowed extracted from the frequency-domain signal; said frequency-domain parameter being selected from a group consisting of total power (TP), high frequency power (HF), low frequency power (LF), very low frequency power (VLF), ultra low frequency power (ULF), low frequency proportion (LF %), and LF/HF ratio.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a first stereo diagram of a contactless physiological measurement device according to the present invention;

FIG. 2 shows a second stereo diagram of the contactless physiological measurement device;

FIG. 3 shows a block diagram of a memory and a processor;

FIG. 4 shows a diagram for describing how to use the contactless physiological measurement device;

FIG. 5 shows a waveform diagram of a first rPPG signal and a second rPPG signal; and

FIG. 6 shows a flowchart of a contactless physiological measurement method according to the present invention.

### DETAILED DESCRIPTION OF THE PREFERRED EMBODIMENTS

To more clearly describe a contactless physiological measurement device and method according to the present invention, embodiments of the present invention will be described in detail with reference to the attached drawings hereinafter.

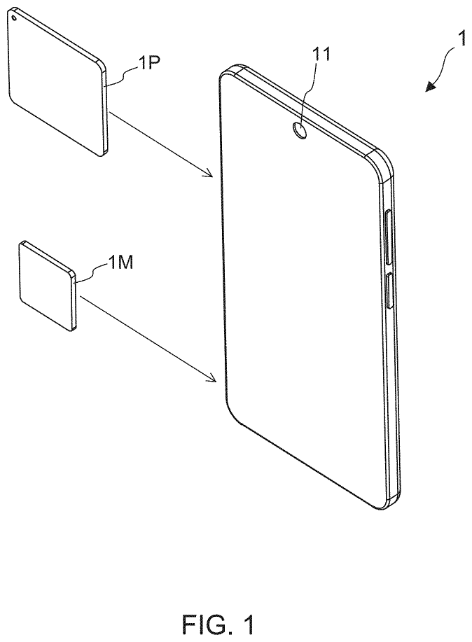

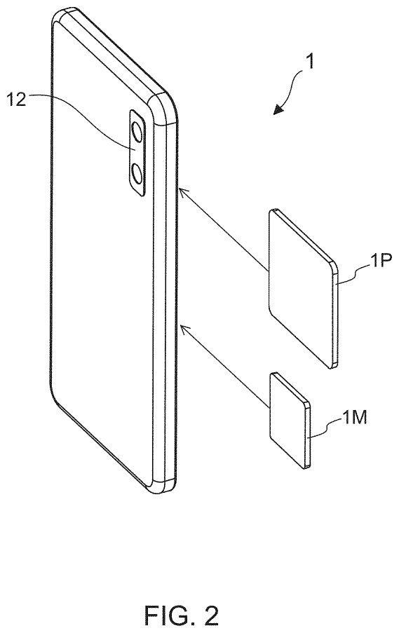

With reference to FIG. 1, there is shown a first stereo diagram of a contactless physiological measurement device according to the present invention. Moreover, FIG. 2 illustrates a second stereo diagram of the contactless physiological measurement device. As FIG. 1 and FIG. 2 shows, the present invention discloses a contactless physiological measurement device 1, which is provided in a form of an electronic device, e.g., smart phone, tablet computer, laptop computer, and all-in-one computer, and comprises a front camera 11, a rear camera 12, a memory 1M, and a processor 1P. As described in more detail below, the processor 1P is coupled to the memory 1M, and is also coupled to the front camera 11 and the rear camera 12.

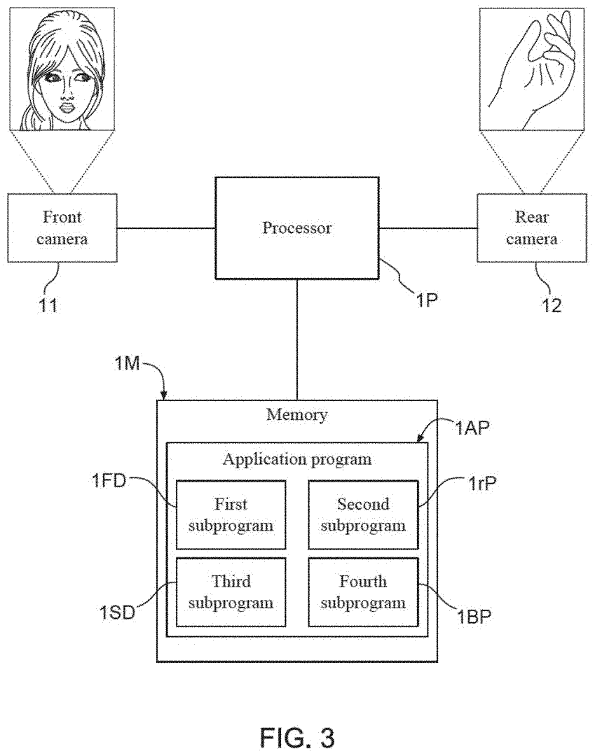

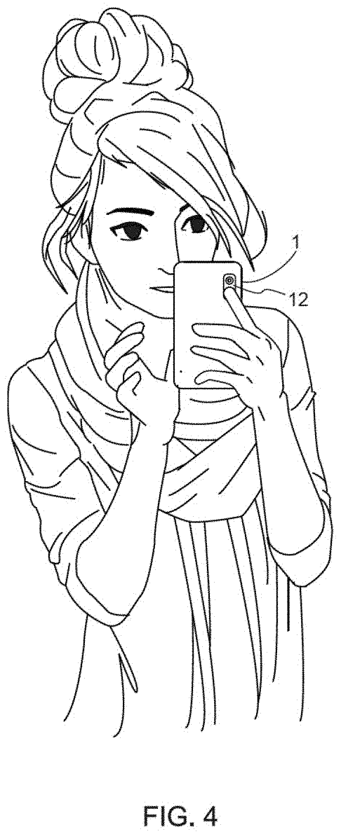

FIG. 3 shows a block diagram of the memory 1M and the processor 1P, and FIG. 4 shows a diagram for describing how to use the contactless physiological measurement device 1. Particularly, there is an application program 1AP stored in the memory 1M, and the application program 1AP comprises: a first subprogram 1FD, a second subprogram 1rP, a third subprogram 1SD, and a fourth subprogram 1BP. By such arrangements, in case the application program 1AP is executed, the processor 1P firstly controls the front camera 11 and the rear camera 12 to photograph a user, so as to acquire at least one first image frame containing a face image and at least one second image frame containing a hand image. For example, as FIG. 4 shows, the contactless physiological measurement device 1 is provided in a form of smart phone, and is held in left hand of a user. Moreover, the front camera 11 of the smart phone is faced to the front of the user, and the rear camera 12 of the smart phone is faced to an index finger of the user's left hand. In such case, the front camera 11 is controlled (by the processor 1P) to acquire at least one first image frame from the front of the user, and the rear camera 12 is controlled to acquire at least one second image frame from the left hand of the user, such that said first image frame contains a (user) face image, and said second image frame contains a hand image.

As explained in more detail below, the first subprogram 1FD is compiled to be integrated in the application program 1AP by one type of programming language, and includes instructions for configuring the processor 1P to perform a face detection function, thereby detecting the face image from the first image frame.

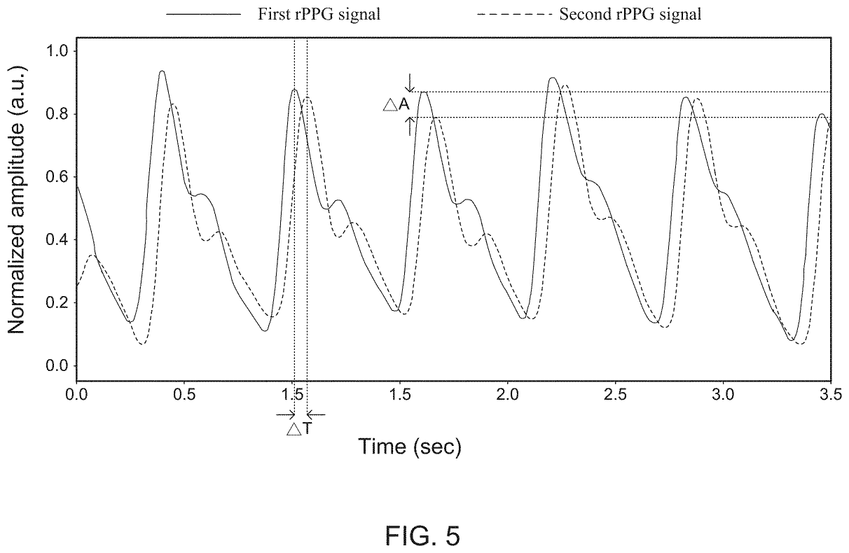

In the execution of the application program 1AP, the processor 1P subsequently extract a first rPPG signal from the face image of said first image frame and a second rPPG signal from the hand image of said second image frame. In one embodiment, the second subprogram 1rP is also compiled to be integrated in the application program 1AP by one type of programming language, and includes instructions for configuring the processor 1P to extract the first rPPG signal and the second rPPG signal from the face image of said first image frame and the hand image of said second image frame, respectively. FIG. 5 shows a waveform diagram of the first rPPG signal and the second rPPG signal.

According FIG. 3 and FIG. 4, the processor 1P subsequently applies a signal difference calculation to the first rPPG signal and the second rPPG signal, so as to calculate at least one signal parameter difference between the two rPPG signals. In FIG. 5, said signal parameter difference is labeled to ΔT and/or ΔA, where ΔT means a pulse interval time (PT), and AA represents a pulse amplitude difference. As explained in more detail below, in a practicable embodiment, the third subprogram 1SD is compiled to be integrated in the application program 1AP by one type of programming language, and including instructions for configuring the processor 1P to perform the function of calculating the at least one signal parameter difference between the first rPPG signal and the second rPPG signal.

In the execution of the application program 1AP, the processor 1P subsequently calculates a plurality of physiological parameters by applying a signal process to the first rPPG signal or the second rPPG signal, wherein the plurality of physiological parameters comprise, but are not limit to, heart rate (HR), heart rate variability (HRV), respiratory rate, and blood oxygen saturation (SpO2).

In one practicable embodiment, a time-domain signal is generated after applying said signal process to the first rPPG signal or the second rPPG signal, such that at least one time-domain parameter is allowed extracted from the time-domain signal. Said time-domain parameter is adopted in the calculation of heart rate variability (HRV), and can be any one of, but is not limit to, standard deviation of all normal to normal intervals (SDNN), root mean square successive differences (RMSSD), number of pairs of adjacent normal to normal intervals differing by more than 50 ms (NN50), and proportion of NN50 divided by a total number of all normal to normal intervals (pNN50).

In another one practicable embodiment, a frequency-domain signal is generated after applying said signal process to the first rPPG signal or the second rPPG signal, such that at least one frequency-domain parameter is allowed extracted from the frequency-domain signal. Said frequency-domain parameter can be, but is not limit to, total power (TP), high frequency power (HF), low frequency power (LF), very low frequency power (VLF), ultra low frequency power (ULF), low frequency proportion (LF %), or LF/HF ratio. Herein, it is worth mentioning that, literature 1 has fully introduced the relationship between TP, HF, LF, VLF, ULF, and/or LF % and the physiological parameters. Literature 1, written by F. Shaffer et al, is entitled with “An Overview of Heart Rate Variability Metrics and Norms” and has DOI:10.3389/fpubh.2017.00258.

Eventually, the instructions included in the application program 1AP configure the processor 1P to input at least one user anthropometric parameter, at least one said physiological parameter and said signal parameter difference into a pre-trained blood pressure estimating model, such that the pre-trained blood pressure estimating model outputs an estimation value of blood pressure. Herein, said user anthropometric parameter can be, but is not limit to, weight, height, body mass index (BMI) and age. As explained in more detail below, the fourth subprogram 1BP is compiled to be integrated in the application program by one type of programming language, and includes instructions for configuring the processor 1P to perform the function of inputting said user anthropometric parameter, said signal parameter difference and at least one said physiological parameter into the pre-trained blood pressure estimating model, thereby outputting said estimation value of blood pressure.

In addition, according to FIG. 3, the memory 1M can be arranged to comprises a database storing at least one user anthropometric data and the plurality of physiological parameters.

Therefore, through the above descriptions, all embodiments of the contactless physiological measurement device 1 according to the present invention have been introduced completely and clearly. Moreover, the present invention simultaneously discloses a contactless physiological measurement method. As FIG. 1, FIG. 2 and FIG. 3 show, the method is compiled to be an application program 1AP so as to be stored in a memory 1M of an electronic device, and is conducted by a processor 1P of the electronic device.

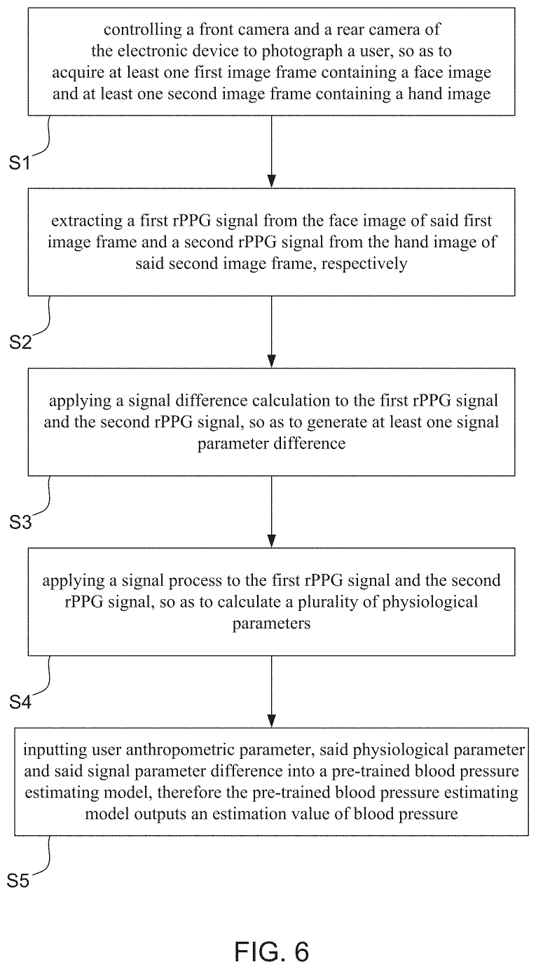

FIG. 6 illustrates a flowchart of a contactless physiological measurement method according to the present invention. According to FIG. 6, the contactless physiological measurement method comprising the steps of:
S1: acquiring, by controlling a front camera 11 and a rear camera 12 of the electronic device 1 to photograph a user, at least one first image frame containing a face image and at least one second image frame containing a hand image;
S2: extracting, from the face image of said first image frame and the hand image of said second image frame, respectively, a first rPPG signal and a second rPPG signal;
S3: calculating, by applying a signal difference calculation to the first rPPG signal and the second rPPG signal, at least one signal parameter difference;
S4: calculating, by applying a signal process to the first rPPG signal and the second rPPG signal, a plurality of physiological parameters; and
S5 outputting, by inputting at least one user anthropometric parameter, at least one said physiological parameter and said signal parameter difference into a pre-trained blood pressure estimating model, an estimation value of blood pressure.

Therefore, above descriptions have introduced the contactless physiological measurement device and method according to the present invention completely and clearly. Moreover, the above description is made on embodiments of the present invention. However, the embodiments are not intended to limit the scope of the present invention, and all equivalent implementations or alterations within the spirit of the present invention still fall within the scope of the present invention.

What is claimed is:

1. A contactless physiological measurement device, being provided in a form of an electronic device having a front camera and a rear camera, and comprising:
a memory storing an application program; and
a processor, being coupled to the memory, and being also coupled to the front camera and the rear camera; wherein the application program includes instructions, such that in case the application program is executed, the processor being configured for:
acquiring, by controlling the front camera and the rear camera to photograph a user, at least one first image frame containing a face image and at least one second image frame containing a hand image;
extracting, from the face image of said first image frame and the hand image of said second image frame, respectively, a first rPPG signal and a second rPPG signal;
calculating, by applying a signal difference calculation to the first rPPG signal and the second rPPG signal, at least one signal parameter difference;
calculating, by applying a signal process to the first rPPG signal and the second rPPG signal, a plurality of physiological parameters; and
outputting, by inputting at least one user anthropometric parameter, at least one said physiological parameter and said signal parameter difference into a pre-trained blood pressure estimating model, an estimation value of blood pressure.

2. The contactless physiological measurement device of claim 1, wherein the electronic device is selected from a group consisting of smart phone, tablet computer, laptop computer, and all-in-one computer.

3. The contactless physiological measurement device of claim 1, wherein the plurality of physiological parameters comprises heart rate (HR), heart rate variability (HRV), respiratory rate, and blood oxygen saturation (SpO2).

4. The contactless physiological measurement device of claim 1, wherein said user anthropometric parameter is selected from a group consisting of weight, height, body mass index (BMI) and age.

5. The contactless physiological measurement device of claim 1, wherein the memory comprises a database storing at least one user anthropometric data and the plurality of physiological parameters.

6. The contactless physiological measurement device of claim 1, wherein the application program consists of a plurality of subprograms, and the plurality of subprograms comprising:
a first subprogram, being compiled to be integrated in the application program by one type of programming language, and including instructions for configuring the processor to detect the face image from the first face image frame;
a second subprogram, being compiled to be integrated in the application program by one type of programming language, and including instructions for configuring the processor to extract the first rPPG signal and the second rPPG signal from the face image of said first image frame and the hand image of said second image frame, respectively;
a third subprogram, being compiled to be integrated in the application program by one type of programming language, and including instructions for configuring the processor to apply said signal difference calculation to the first rPPG signal and the second rPPG signal, thereby calculating the at least one signal parameter difference; and
a fourth subprogram, being compiled to be integrated in the application program by one type of programming language, and including instructions for configuring the processor to input said user anthropometric parameter, said signal parameter difference and at least one said physiological parameter into the pre-trained blood pressure estimating model, such that the pre-trained blood pressure estimating model outputs said estimation value of blood pressure.

7. A contactless physiological measurement method, being compiled to be an application program so as to be stored in a memory of an electronic device, and being conducted by a processor of the electronic device; the contactless physiological measurement method comprising the steps of:
(1) acquiring, by controlling a front camera and a rear camera of the electronic device to photograph a user, at least one first image frame containing a face image and at least one second image frame containing a hand image;
(2) extracting, from the face image of said first image frame and the hand image of said second image frame, respectively, a first rPPG signal and a second rPPG signal;
(3) calculating, by applying a signal difference calculation to the first rPPG signal and the second rPPG signal, at least one signal parameter difference;
(4) calculating, by applying a signal process to the first rPPG signal and the second rPPG signal, a plurality of physiological parameters; and
(5) outputting, by inputting at least one user anthropometric parameter, at least one said physiological parameter and said signal parameter difference into a pre-trained blood pressure estimating model, an estimation value of blood pressure.

8. The contactless physiological measurement method of claim 7, wherein the electronic device is selected from a group consisting of smart phone, tablet computer, laptop computer, and all-in-one computer.

9. The contactless physiological measurement method of claim 7, wherein said user anthropometric parameter is selected from a group consisting of weight, height, body mass index (BMI) and age.

10. The contactless physiological measurement method of claim 7, wherein the plurality of physiological parameters comprises heart rate (HR), heart rate variability (HRV), respiratory rate, and blood oxygen saturation (SpO2).

11. The contactless physiological measurement method of claim 7, wherein the memory comprises a database storing at least one user anthropometric data and the plurality of physiological parameters.

12. The contactless physiological measurement method of claim 7, wherein the application program consists of a plurality of subprograms, and the plurality of subprograms comprising:
a first subprogram, being compiled to be integrated in the application program by one type of programming language, and including instructions for configuring the processor to detect the face image from the first image frame;
a second subprogram, being compiled to be integrated in the application program by one type of programming language, and including instructions for configuring the processor to extract the first rPPG signal and the second rPPG signal from the face image of said first image frame and the hand image of said second image frame, respectively;
a third subprogram, being compiled to be integrated in the application program by one type of programming language, and including instructions for configuring the processor to apply said signal difference calculation to the first rPPG signal and the second rPPG signal, thereby calculating the at least one signal parameter difference; and
a fourth subprogram, being compiled to be integrated in the application program by one type of programming language, and including instructions for configuring the processor 1P to input said user anthropometric parameter, said signal parameter difference and at least one said physiological parameter into the pre-trained blood pressure estimating model, such that the pre-trained blood pressure estimating model outputs said estimation value of blood pressure.
